(19) United States
(12) Patent Application Publication
(10) Pub. No.: US 2022/0346641 A1
(43) Pub. Date: Nov. 3, 2022

(54) PUPILLARY DISTANCE MEASURING DEVICE

(71) Applicant: Chioma Michaelson, Lawrenceville, GA (US)

(72) Inventor: Chioma Michaelson, Lawrenceville, GA (US)

(21) Appl. No.: 17/243,838

(22) Filed: Apr. 29, 2021

(51) Int. Cl. A61B 3/11; A61B 3/00

(52) U.S. Cl. CPC A61B 3/111; A61B 3/0083

(57) ABSTRACT

Embodiments of the present disclosure relate generally to a pupillary distance measuring device. The device is designed so that it can be used in a home-based or other non-professional/non-doctor office environment. The individual can use the device to check his/her pupillary distance measurement and then purchase eyeglasses online, without the need for a professional optician's assistance.

### FIELD OF THE DISCLOSURE

Embodiments of the present disclosure relate generally to a pupillary distance measuring device. The device is designed so that it can be used in a home-based or other non-professional/non-doctor office environment. The individual can use the device to check his/her pupillary distance measurement and then purchase eyeglasses online, without the need for a professional optician's assistance.

### BACKGROUND

Prescription optical lenses have a center point called the “optical center,” at which point the power is 100% as prescribed. The best vision is obtained through glasses when the optical center of the lens is placed directly over the center of the pupil. In order to identify the optical center, a practitioner typically measures the distance between the centers of a patient's pupils. This measurement is called the inter-pupillary distance or pupillary distance (“PD”). If the optical center of glasses does not match the centers of the patient's pupils, vision can be affected—and it can feel like wearing someone else's glasses. Even a small PD error can make prescription eyeglasses exhibit prismatic behavior, which can cause squinting, eyestrain, fatigue, blurry vision, headaches, and/or dizziness. These symptoms can be exacerbated at higher prescriptions with the wrong PD. Aligning an optical center of the lens with the individual's pupil is thus a key factor for making prescription eyeglasses successfully.

There are two types of PDs that can be measured. The first type is binocular PD, in which the actual distance between the centers of the two pupils is measured. This is illustrated by FIG. 7. The second type is monocular PD, which is measured from the center of one pupil to the center of the bridge of the nose, calculating individuals for the left and right PD. In other words, one value is calculated for the distance from the center of the left pupil to the center of the bridge of the nose, and another value calculated for the distance from the center of the right pupil to the center of the bridge of the nose. This is illustrated by FIG. 8. Monocular PD is more accurate because eyes are not always equally centered the same distance from the bridge of the nose on the left and the right side of the face. Monocular PD can adjust for this potential imbalance. For example, a patient's binocular PD may be 68 mm. However, that does not mean that the left PD is 34 mm and the right PD is 34 mm. Instead, the measurements could be 35/33, 33/35, 36/32, 32/36 or even further varied.

Traditionally, measuring PD has typically required individuals to use the services of an optician (at a brick-and-mortar vision center) with specialized equipment in order to obtain these measurements. Vision clinics normally measure the PD using either a digital pupillometer, as shown by FIGS. 10 and 11. Not all opticians or eye doctors include the PD measurement in the written eyeglasses prescription. Also, because opticians purchase specialized PD meter equipment, they may not be willing to give free PD readings to patients who choose to purchase their eyeglasses elsewhere. However, if a patient wishes to purchase eyeglasses online, s/he will need to provide the glasses maker with a PD measurement, as well as the lens prescription.

Several non-empirical methods of PD measurements are suggested online and include using a credit card to guess one's PD. Other systems provide PD rulers that can sit on the bridge of the nose. Further systems provide various other types of sliding ruler systems. Most of the current inventions all lean toward providing digital measurements, which can be confusing for an at-home patient. Additionally, these methods typically provide a binocular PD distance (the single measurement between both pupils), not the monocular left and right PD distances that are necessary for a successful fit. Further improvements to pupillary distance measuring are thus desirable.

### BRIEF SUMMARY

Embodiments of the present disclosure relate generally to a pupillary distance measuring device. The device is designed so that it can be used in a home-based or other non-professional/non-doctor office environment. The individual can use the device to check his/her pupillary distance measurement and then purchase eyeglasses online, without the need for a professional optician's assistance.

Embodiments of the present disclosure provide a pupillary distance (PD) measuring device, comprising a face frame comprising a bridge and side temples, the face frame comprising an upper bar with a plurality of distance markings starting at zero at the middle of the frame over the nose area, wherein the distance markings accommodate for eye convergence by starting measurement points about at increase of about 1.5 mm higher than actual measurement on both the right and left side with two sliding markers that are slidable along both sides of the upper bar.

### BRIEF DESCRIPTION OF THE DRAWINGS

FIG. 1 shows a front view of a measuring device according to one embodiment of the disclosure with the sliding marker in one position.

FIG. 2 shows a front view of a measuring device according to one embodiment of the disclosure with the sliding marker in a different position.

FIG. 3 shows a schematic view of a sliding marker.

FIG. 4 shows a schematic view of exemplary distance markings.

FIG. 5 shows a perspective view of eclipse glasses that may form a face frame portion of a measuring device according to one embodiment of the disclosure.

FIG. 6 shows a perspective view of a plastic glasses frame that may form a face frame portion of a measuring device according to one embodiment of the disclosure.

FIG. 7 shows a schematic of binocular pupillary measurement.

FIG. 8 shows a schematic of monocular pupillary measurement.

FIG. 9 shows one embodiment of a clip-on device according to this disclosure.

FIG. 10 shows a schematic view of one embodiment of a prior art pupillometer.

FIG. 11 shows a side perspective view of a user of a pupillometer of FIG. 10.

### DETAILED DESCRIPTION

Embodiments of the present disclosure simplify the principles used with digital pupillometer and PD ruler methods. The disclosed PD measurement device also incorporates a professional fact that is not immediately obvious to even members of the optical profession. Digital pupillometers accommodate for convergence of the eyes when the patient is changing his/her gazing point of reference from far to near. The eyes converge (move slightly inward) when the point of reference is close in range, which can mean that the pupils are closer together when the patient is viewing something close up. There is typically a 3 mm difference between PD measurement when a patient is looking near/close-up versus far/distance. This can directly (and negatively) affect a patient's use of a home-based PD measurement device that does not accommodate for this type of convergence, particularly because patients use these at close range.

Accordingly, the disclosed PD measurement device provides its measurement bar with an initial addition of 1.5 mm on each of the left and right sides of the measuring device. This pre-compensates for the total of a 3 mm convergence, while still measuring the PD distance at near on each side.

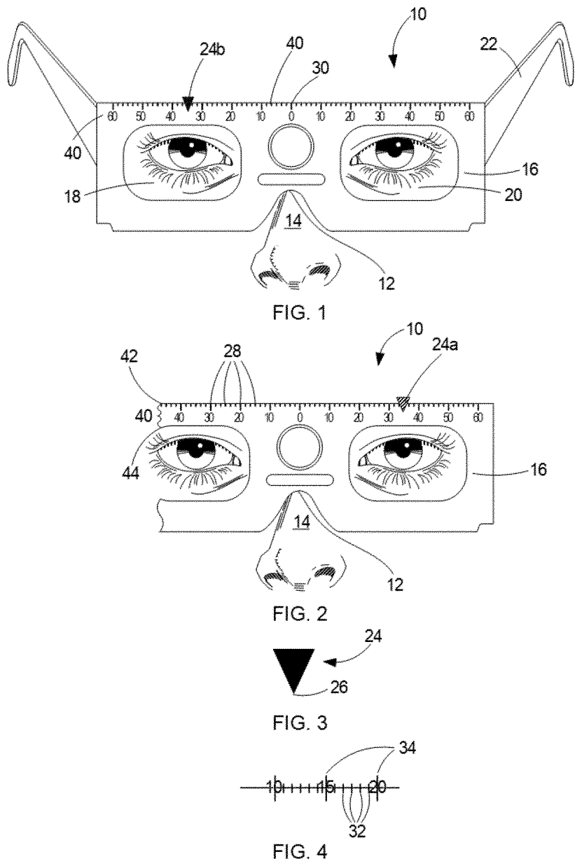

Referring now to FIG. 1, there is shown one embodiment of a measuring device 10 described herein. The measuring device 10 can be worn like a pair of eyeglasses, such that a bridge 12 of the device sits on a user's nose bridge 14. The device 10 has a face frame portion 16 with two openings 18, 20 for the user's eyes. The openings can, but need not have any lens or material therein. They may simply be provided as holes in the face frame portion 16, much like holes in a glasses frame before lenses are inserted. Although not required, the device 10 may also have side temples 22, similar to traditional eyeglasses temples, which are curved and hook over a user's ears.

Positioned along an upper edge 18 of the face frame portion 16 is at least one sliding marker 24. One example is illustrated by FIG. 3. In one example, the sliding marker 24 may have a pointed end 26, much like a triangle point. This can make it easier for a user to align the pointed end 26 directly with the center of his/her pupil, as illustrated by FIGS. 1 and 2. Alternatively, it is possible for any appropriate indicator to be used on sliding marker 24, such as an arrow, a pinpoint, or any other appropriate indicator.

In one example, it is possible to provide a first slide marker 24a that is positioned on the temporal side of the right side of the frame and a second slide marker 24b on the temporal side of the left side of the frame. These slide markers 24a, 24b may be colored differently in order to make recordation of the readings easier for the user. Providing two slide markers 24a, 24b can also allow both sides to be measured while the measuring device 10 remains on the user's face, as described in more detail by the below methods.

An upper bar 40 of the face frame portion 16 also includes distance markings 28. These distance markings 28 are generally provided in millimeters (mm), although it is possible for other measurement units to be used. (Millimeters are most traditionally used in the optical field to measure pupillary distance.) A central point 30 on the face frame portion 16 marks the center of the frame bridge 12. Extending out on left and right hand sides are distance markings 28 indicating measurements marked in millimeters. Measurements may be marked by a plurality of measurement points. Each measurement point may be generally marked by a hash line 32. For example, each single millimeter may be marked with a hash line 32. Similar to a ruler, millimeters in units of 5 or 10 may be marked by a plurality of measurement have more elongated hash lines 34. A close up example is illustrated by FIG. 4. Use of periodic elongated hash lines 34 in this way can make it easier for the user to identify and count the measurement. The distance markings 28 may be provided only along the upper edge 42 of the upper bar 40. The distance markings 28 may be provided only along the lower edge 44 of the upper bar 40. It is also possible to provide distance markings 28 along both the upper edge 42 and the lower edge 44 of the upper bar 40 of the face frame portion 16. The length of the upper bar 40 (temple length) may be about 140-150 mm. It should be understood, however, that larger or smaller sizes may be provided, such as a child-sized measuring device with a temple length of about 120-130 mm.

The sliding marker(s) 24 is/are generally positioned so it/they can slide across the upper bar 40. The sliding marker(s) 24 may be a small plastic clip component with an internal channel that receives the upper bar 40. In a specific example, there are two sliding markers provided. The sliding markers may be color-coded or otherwise marked to make recording the PD measurements easier for the user. For example, the sliding marker over the right eye may be red and the sliding marker over the left eye may be blue (or vice versa). Additionally or alternatively, differently patterned sliding markers may be used.

In use, the user wears the measuring device 10 like a pair of glasses. It is optimal to conduct the described method in a well-lit location. The user then measures one of either the right or left PD, will make note of that side's PD, and then conducts the same steps to measure the other of the right or left PD, and makes a note of that side's PD. Alternatively, if two sliding markers are provided, both the left and the right PD can be measured while the measuring device 10 remains positioned on the user's face, and both measurements can be noted once the measuring device 10 is removed. In this version, there are two sliding markers, and PD measurements are taken of both eyes by each sliding marker, one after the other.

In one example, if the right PD is to be measured first, the user positions the measuring device 10 on his/her face, with the bridge 12 resting on his/her nose bridge 14. The user then looks into a mirror or other reflecting device. For obtaining the right hand side measurement, the user should look directly into his/her right eye in the mirror or other reflecting device. For obtaining the left hand side measurement, the user should look directly into his/her left eye in the mirror or other reflecting device. (If the measurement is being done by someone other than the person wearing the glasses, such as a parent measuring a child's PD, the person wearing the glasses should look at the nose (or other central point) on the person doing the measuring while each eye is being measured.

In order to begin measuring, the user should look directly into the eye being measured (e.g., either the right eye or the left eye), move the sliding marker over to pupil, and align the pointed end 26 of the sliding marker 24 with the center of the pupil. The same steps are then conducted on the opposite side.

Because the user is typically conducting this step in a mirror and because eyes typically converge differently when looking close up versus far away as described above, the distance markings 28 are numbered so that they are not the exact distances, but are instead numbered in order to accommodate for such convergence. Accordingly, on the right-hand side, instead of marking the 10 mm distance from the device bridge 12 as “10 mm,” the 11.5 mm distance is marked as 10 mm, in order to accommodate for half (1.5 mm) of above-discussed 3 mm convergence. Similarly, on the left-hand side, instead of marking the 10 mm distance from the device bridge 12 as “10 mm,” the 11.5 mm distance is marked as 10 mm, in order to accommodate for other half (the other 1.5 mm) of above-discussed 3 mm convergence. Each side is then numbered out past at least 45 mm, and may generally extend out to about 60 or 70 mm in order to accommodate for differently-sized faces. This is illustrated by FIGS. 1 and 2.

User instructions may be printed on the inside of one of the temples or be provided with packaging materials. Below is a set of exemplary user instructions:
User Instruction: Do not use if slider is broken. Do not use if the ruler markings on the front are faded or missing. These are not eclipse glasses, do not gaze at the sun with these glasses. These are not prescription glasses.
a. Put your glasses on in front of a mirror and adjust on face and ears with the bridge of the glasses resting on the bridge of your nose.
b. Look directly into your right eye, with your right hand, slide the red bar on your right temple side to the middle till it is over the center of your right pupil, stop.
c. Look directly into your left eye, with your left hand, slide the blue bar on your left temple side to the middle till it is over the center of your pupil, stop.
d. Take your glasses off and check the readings on the PD Meter. Record the RE reading for the right eye. Record the LE reading for your left eye.
e. Note the date. Children grow daily, remeasure PD every year.
f.

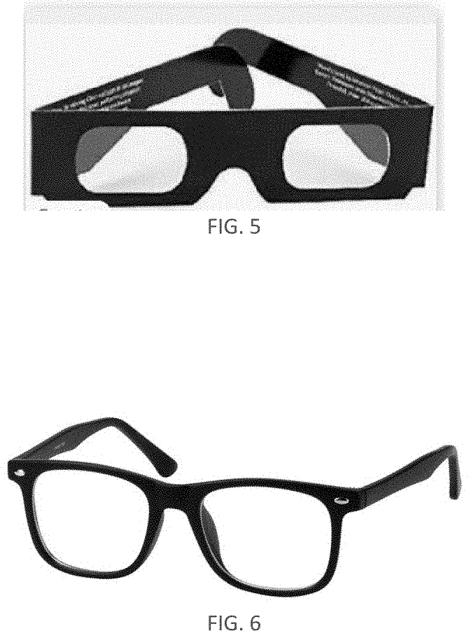

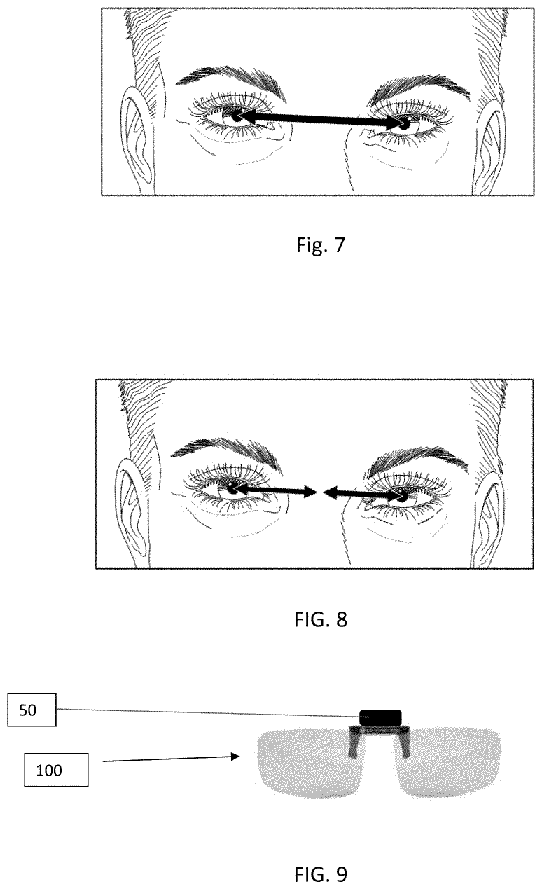

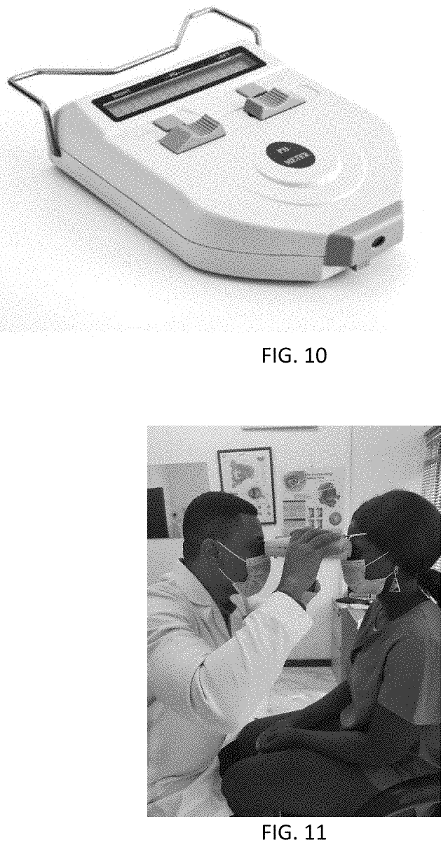

The material of the measuring device 10 may be paper, similar to eclipse-watching paper glasses. One example is illustrated by FIG. 5. This can be a highly economical option. In other embodiments, the material of the measuring device 10 may be plastic, similar to inexpensive reader-type glasses. One example is illustrated by FIG. 6. This option can provide more stability to the face frame portion 16 and other components.

The below Table of Technical Information provides one specific and exemplary embodiment of a PD measuring device according to this disclosure. It should be understood, however, that these dimensions are provided as an example only, and different (larger and smaller) dimensions may be used in a device manufactured according to this disclosure.

TABLE 1
TECHNICAL INFORMATION

Front of adult PD Meter is 143 mm
70 mm + 70 mm + C
(C = 3 mm)
= 143 mm
Front children’s PD Meter is 123 mm
(60 mm + 60 mm + C
(C = 3 mm)
= 123 mm
Adult Temple Length to Bend = 140 mm with option to lengthen
Child Temple Length to Bend = 110 mm with option to lengthen

In one example, the top of the glasses may be marked from a point slightly off center of both sides to both temples in millimeters. The mark may begin at zero+C to 45 mm on both sides for adults and zero+C to 35 mm on both sides for children.

In a further embodiment, the PD measuring device may be provided as a clip-on device 100 that can be clipped over a user's existing eyeglasses. One embodiment is illustrated by FIG. 9 (with the PD measurements and sliding maker(s) not shown for simplicity.) This clip on version would have all of the above-described features and is used similarly, except that temples 22 may be omitted and one or more clips 50 may be added along upper or side edges of the clip-on device in order to secure the clip-on device 100 to a user's existing eyeglasses.

The subject matter of certain embodiments of this disclosure is described with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

It should be understood that different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Changes and modifications, additions and deletions may be made to the structures and methods recited above and shown in the drawings without departing from the scope or spirit of the invention disclosure and the following claims.

What is claimed is:

1. A pupillary distance (PD) measuring device, comprising:
a face frame comprising a bridge;
the face frame comprising an upper bar with a plurality of distance markings, wherein the distance markings accommodate for eye convergence by starting measurement points about at increase of about 1.5 mm higher than actual measurement;
at least one sliding marker slidable along the upper bar.

2. The device of claim 1, wherein a measurement point that is actually 10 mm from a centerline of the bridge is marked as 11.5 mm.

3. The device of claim 1, wherein the plurality of distance markings are positioned along an upper edge of the upper bar, along a lower edge of the upper bar, or both.

4. The device of claim 1, wherein the at least one sliding marker comprises a first sliding marker and a second sliding marker.

5. The device of claim 4, wherein the first and second sliding markers are differently colored.

6. The device of claim 1, wherein the measurement points comprise hash lines.

7. The device of claim 1, further comprising temples.

8. The device of claim 1, wherein the device is a clip-on device with one or more clips (50) that allow the device to be clipped onto a user's eyeglasses.

9. A method for self-measuring a user's pupillary distance, comprising:
positioning the device of claim 1 on the user's face with the bridge resting on the user's nose bridge;
while looking into a mirror or other reflective surface:
looking directly into the right eye and sliding the at least one sliding marker until it is over the center of the right pupil,
looking directly into the left eye and sliding the at least one sliding marker until it is over the center of the left pupil,
notating the distance markings number of the sliding marker for each measurement.

10. The method of claim 9, wherein the device comprises first and second sliding markers, wherein both right and left measurements are made while the user is wearing the device, and wherein the distance markings are notated once the device is removed.

11. A method for measuring a user's pupillary distance, comprising:
positioning the device of claim 1 on the user's face with the bridge resting on the user's nose bridge;
sliding the at least one sliding marker until it is over a center of the user's right pupil;
sliding the at least one sliding marker until it is over a center of the user's right pupil;
notating the distance markings number of the sliding marker for each measurement.
